(19) United States
(12) Plant Patent Application Publication
(10) Pub. No.: US 2005/0183181 P1
(43) Pub. Date: Aug. 18, 2005

(54) Chrysanthemum plant named 'Peach Spider'

(50) CHRYSANTHEMUM MORIFOLIUM; CHRYSANTHEMUM

(76) Inventor: Keith G. Lintott, Bognor Regis (GB)

Correspondence Address: Mark K. Dickson, Suites 200 & 300, 2941 Fairview Park Drive, Falls Church, VA, 22042, US

(21) Appl. No.: 11/055,216

(22) Filed: Feb. 9, 2005

Related U.S. Application Data
(60) Provisional application No. 60/544,383, filed on Feb. 12, 2004.

(51) Int. Cl. A01H 5/00

(52) U.S. Cl. PLT/291

(57) ABSTRACT

A new variety of Chrysanthemum named ‘Peach Spider,’ having a domed capitulum of peach colored spider type decorative flowers and a round shape. The flowers last well under adverse weather conditions of wind and rain.

This application claims the benefit of U.S. Provisional Application No. 60/544,383, filed on Feb. 12, 2004.

### LATIN NAME OF THE GENUS AND SPECIES

Botanical classification: Chrysanthemum morifolium.

### VARIETY DENOMINATION

The new Chrysanthemum variety denomination is ‘Peach Spider.’

### BACKGROUND OF THE INVENTION

The present invention comprises a new and distinct cultivar of Chrysanthemum botanically known as Chrysanthemum morifolium, and referred to by the variety name ‘Peach Spider.’

‘Peach Spider,’ identified as 30853-0, originated from a cross, made in a controlled breeding program in Chichester, West Sussex, United Kingdom.

The female parent of the new Chrysanthemum variety is the variety known as ‘Sarah’ U.S. Plant Pat. No. 7586. The male parent of the new Chrysanthemum variety is an undistributed, unpatented seedling identified as ‘G26C23.’ The new Chrysanthemum variety ‘Peach Spider’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Peach Spider’ is a garden type of Chrysanthemum plant variety having a domed capitulum of peach colored quilled decorative flowers.

### Comparison with Female Parent

Plants of the new Chrysanthemum variety ‘Peach Spider’ are dissimilar to the parent variety ‘Sarah’ in plant habit and growth rate. In side-by-side comparisons in Chichester, West Sussex, United Kingdom, under commercial practice, plants of the new Chrysanthemum variety ‘Peach Spider’ differ from plants of the parent variety ‘Sarah’ in the following characteristics.

1. The new Chrysanthemum variety ‘Peach Spider’ produces peach colored quilled petal type decorative flowers whereas the parent variety ‘Sarah’ produces bronze colored quilled decorative type flowers.

2. Plants of the new Chrysanthemum variety ‘Peach Spider’ have larger and longer inflorescence than plants of the parent variety ‘Sarah.’

3. Plants of the new Chrysanthemum variety ‘Peach Spider’ have a more branching and spreading habit than plants of the parent variety ‘Sarah.’

### Comparison with Male Parent

Plants of the new Chrysanthemum variety ‘Peach Spider’ are similar to the male parent variety ‘G26C23’ in plant habit and growth rate. However, in side-by-side comparisons in Chichester, West Sussex, United Kingdom, under commercial practice, plants of the new Chrysanthemum variety ‘Peach Spider’ differed from plants of the male cultivar ‘G26C23’ in the following characteristics:

1. The new variety ‘Peach Spider’ produces peach colored quilled petal type decorative flowers whereas the male parent variety ‘G26C23’ produces pink colored quilled petal type decorative flowers.

2. The new variety ‘Peach Spider’ produces a more rounded and domed plant, than the male parent variety ‘G26C23.’

3. The new variety ‘Peach Spider’ flowers near 9 weeks natural season whereas plants of the male parent flower near 10 weeks natural season.

### Comparison with the Variety ‘Yellow Sarah’

Plants of the new Chrysanthemum variety ‘Peach Spider’ are dissimilar to the variety ‘Yellow Sarah,’ U.S. Plant Pat. No. 8,804, in plant habit and growth rate. In side-by-side comparisons in Chichester, West Sussex, United Kingdom, under commercial practice, plants of the new Chrysanthemum variety ‘Peach Spider’ differed from the variety ‘Yellow Sarah’ in the following characteristics:

1. Plants of the new Chrysanthemum variety ‘Peach Spider’ produce peach colored quilled petal type decorative flowers whereas plants of the variety ‘Yellow Sarah’ produce yellow quilled petal type decorative flowers.

2. Plants of the new Chrysanthemum variety ‘Peach Spider’ have a more rounded and domed habit than plants of the variety ‘Yellow Sarah.’

3. Plants of the new Chrysanthemum variety ‘Peach Spider’ have larger and longer inflorescence than plants of the variety ‘Yellow Sarah.’

### BRIEF DESCRIPTION OF ILLUSTRATION

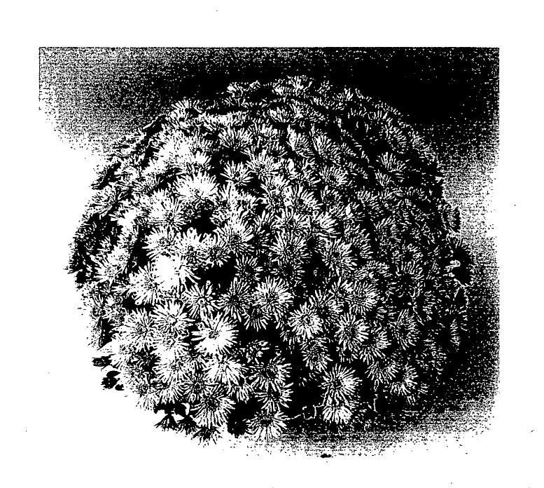

Typical specimens of the plant and flowers for the new Chrysanthemum variety ‘Peach Spider’ are shown in the accompanying digital photograph. The colors shown are as true as possible within the usual limits of this kind of illustration. FIG. 1 is a whole plant view of the new variety ‘Peach Spider’ grown in a pot. The plant shown in the illustration is 63 days from the commencement of planting outdoors in mid-June.

### DETAILED BOTANICAL DESCRIPTION

The following description of the new Chrysanthemum variety ‘Peach Spider’ is of plants grown outside, natural season, in Chichester, West Sussex, United Kingdom in the month of June. The variety has not been observed under all possible environmental conditions. The phenotype may vary significantly with variations in the environment such as temperature, length of day and light intensity, without any variance in genotype. The commercial classification of the new variety is a garden Chrysanthemum.

Plants of the new variety have been grown successfully under temperature conditions averaging about 50° F. at night and about 60° F. to 75° F. during the day under natural light conditions. The plants were grown outdoors in mid-June in 4 liter pots using loamless compost. No growth retardants were used. To produce a commercial product the plants may be pinched once with the center bud removed. The typical container size for commercial growth is 4 liters (8″). It has been observed that the shelf life of the new variety is about 26 days with a response time of about 9 weeks. The new variety is suitable for growth in a temperature range of 50° F. to 75° F.

The new variety may be produced as a pot plant. The following description is with respect to a plant produced as a garden pot plant. In the description of this new Chrysanthemum variety, color values have been taken from The Royal Horticultural Society Colour Chart (R.H.S.C.C.).

### Plant

Plant type: Pot.
Habit: Domed-Round.
Height: 45 cm-55 cm.
Width: 40 cm-55 cm.
Branching characteristics: Free branching and spreading.
Length of lateral branches: 7 cm-15 cm.
Number of breaks from pinch: 7-12.
Stem:
   Length.—6 cm-8 cm.
   Diameter.—0.6 cm-0.8 cm.
   Texture.—Slightly Pubescent and Rough.
   Color.—near 137C.
Response time: 63 days natural season.
Vigor: Medium.
Shelf life: near 26 days.
Disease (susceptibility/resistance observed): None observed to date.
Pest (susceptibility/resistance observed): None observed to date.
Drought and temperature (susceptibility/resistance observed): None observed to date.
Growth retardant type and treatment: No treatment necessary.
The plants were grown for 9 weeks in natural season conditions. Propagation:
   Type.—Vegetative Propagation via Stem Cuttings.
   Time to rooting.—12 days with soil temperatures of 70° F.
   Rooting habit.—Fine and Fibrous.

### Foliage

Number of leaves per lateral branch: 7-15.
Compound or single: Single.
Arrangement of leaves: Alternate.
Shape of leaf: Typically 5 lobed ovate.
Size of leaf: Width: 2 cm-3 cm Length: 2.5 cm-4 cm.
Leaf apex: Acute.
Base: Obtuse.
Attachment: Petioled.
Aspect: Slightly undulating.
Margin: Palmately lobed.
Surface characteristics: Top: Slightly Pubescent Bottom: Slightly Pubescent.
Petiole: Color: near 138A Length: 0.6 cm-1.0 cm. Diameter: 1 cm-0.3 cm.
Venation: Net. Prominent mid vein at underside Color: upper side: near 138B underside: near 138B.
Color: Mature Leaf, upper side: near 137A; under side: near 137B Young Leaf, upper side: near 137A; under side: near 137B.

### Flower

Flower appearance: Matte.
Flower type: Quilled Decorative.
Flower form: Slightly Domed.
Flower shape: Round.
Flowering habit: Cyme.
Number of blossoms per branch: 7-12.
Typical and observed flowering season: August and September natural season.
Inflorescence form: Corymbiform.
Depth of fully expanded blossoms: 1.2 cm-1.8 cm.
Diameter of fully expanded blossoms: 5 cm-7 cm.
Phyllaries:
   Number.—21-25.
   Color.—near 138A.
   Length.—0.6 cm-1.0 cm.
   Width.—0.2 cm-0.4 cm.
   Texture/Appearance.—Shiny/Pubescent.
Peduncle: Peduncle length is shortest at the terminal flower and progressively longer down the stem; longest length is on average 14cm (cm), shortest length is on average 7 cm
   Length.—7 cm-14 cm.
   Diameter.—0.4 cm-0.7 cm.
   Angle from stem.—20°-25°.
   Color.—near 138A.
   Surface.—Pubescent.
   Habit.—Slightly curved.
   Strength.—Strong.
Pedicel:
   Length.—terminal: 7 cm; lateral: 14 cm.
   Diameter.—0.2 cm-0.4 cm.
   Color.—near 137A.
   Surface.—Slightly pubescent.
   Habit.—Slightly curving.
   Strength.—Medium/Strong.
Ray florets:
   Form/shape.—Quilled.
   Texture/appearance.—Matte.
   Number per flower.—175-190.
   Length.—1.0 cm-4.0 cm; Width: 0.1 cm-0.4 cm.
   Apex: Round.
   Base: Tubular.
   Margin: Entire.
Disc florets: None observed.
Fragrance: Slight.
Flower bud (at onset of color):
   Length.—0.6 cm -1.0 cm.
   Diameter.—0.8 cm 1.0 cm.
   Form/shape.—Round/Flattened.
General flower color:
   1. Ray florets, upper side.—Immature: near 22A Mature: near 22B Older/Fading: near 22C.
   2. Ray florets, under side.—Immature: near 22A Mature: near 22B Older/Fading: near 22C.
   3. Bud.—near 139C.
Flower progression with age; Flowers fade slightly with age. The lastingness of bloom is about 26 days.

### Reproductive Organs

Gynoecium present on ray florets only. Ray florets per individual flower:
   Pistil number.—60-75 observed.
   Stigma color.—near 13A.
   Stigma shape.—Forked.
   Style color.—near 144C.
   Style length.—0.2 cm-0.4 cm.
Androecium not observed.
Fruit and seeds: None observed.

1. A new and distinct variety of Chrysanthemum plant, substantially as described and illustrated herein.
